(19) United States
(12) Patent Application Publication
(10) Pub. No.: US 2015/0283351 A1
(43) Pub. Date: Oct. 8, 2015

(54) TRACHEOSTOMY BREATHING MONITOR

(71) Applicants: Frank Castello, Princeton, NJ (US); Gabriel Geraldo, Kearny, NJ (US); Maria Conneran, Portland, OR (US); Brodie Halbfoster, Columbia, MD (US); Bryan Pfister, Newtown, PA (US); Andrew Finer, Somerset, NJ (US)

(72) Inventors: Frank Castello, Princeton, NJ (US); Gabriel Geraldo, Kearny, NJ (US); Maria Conneran, Portland, OR (US); Brodie Halbfoster, Columbia, MD (US); Bryan Pfister, Newtown, PA (US); Andrew Finer, Somerset, NJ (US)

(21) Appl. No.: 14/443,272

(22) PCT Filed: Nov. 15, 2013
(86) PCT No.: PCT/US2013/070449

§ 371 Date: May 15, 2015

Related U.S. Application Data
(60) Provisional application No. 61/726,838, filed on Nov. 15, 2012.

(51) Int. Cl. A61M 16/08; A61B 5/00; A61M 16/04; A61B 5/097; A61B 5/087

(52) U.S. Cl. CPC A61M 16/0841; A61B 5/097; A61B 5/087; A61M 16/047; A61B 5/746; A61M 2205/8206

(57) ABSTRACT

The present invention provides a medical device for monitoring breathing of a patient comprising an adapter with at least one port; a monitor; and at least one tubing connecting the at least one port and the monitor wherein the adapter attached to a tracheostomy tube.

### FIELD OF THE INVENTION

The present invention relates to a portable device that will alert a caregiver if a tracheostomy patient stops breathing from clogged tubes or decannulation.

### BACKGROUND OF THE INVENTION

A tracheotomy is a surgical procedure where an incision is made into the trachea to create a temporary or permanent breathing airway called a tracheostomy. A tracheostomy is performed on a pediatric patient for different reasons; most are to resolve upper respiratory issues. The leading complications of a tracheostomy result in some obstruction to normal breathing such as decannulation or secretions clogging the tracheostomy tube, which can result in death. Care of the patient after a tracheostomy is centered on ensuring the patient has an unobstructed airway. Accordingly, a device with the means to monitor breathing and alert caregivers to an emergency situation is critical to the well-being of tracheostomy patients.

Currently, the only available airflow monitors are incorporated into heavy mechanical ventilators and are only useful when a patient is connected and restricted to the bed. There is no ideal monitor for ensuring airway patency in patients, especially infants and young children with tracheostomies. Typically, pulse oximetry or cardiac monitors are used as a surrogate to detect problems with an occluded or dislodged tube and will alarm only when the patient has either low levels of oxygen in the blood (pulse oximeter) or a low heart rate from lack of oxygen (cardiac monitor). In either case, these monitoring devices are detecting a secondary problem, lack of oxygen, rather than the primary problem of a tracheostomy complication. There is currently no reliable, portable device that directly monitors tracheostomy airflow and alarms when expiratory flow is very low or absent.

Thus there remains a need for an improved airflow monitor that is portable and directly monitors tracheostomy airflow.

### SUMMARY OF THE INVENTION

The present invention relates to a portable device that will alert a caregiver if a tracheostomy patient stops breathing. The monitoring device can be used with any patient that needs monitoring of the airway, including, for example, a human or an animal that has had a tracheostomy.

More particularly, the invention provides a medical device for monitoring breathing of a patient comprising an adapter with at least one port; a monitor; and at least one tubing connecting the at least one port of the adaptor with the monitor, wherein the adapter attaches to a tracheostomy tube.

The monitor comprises an air flow sensor, an electronic control and a battery.

In one embodiment, the monitor further comprises an alarm.

In another embodiment, the medical device is connected to an alarm. The alarm may be attached directly to the monitor or it may not b e directly attached to the monitor.

In a further embodiment, the alarm is controlled remotely.

The adapter of the invention is designed to attach to a tracheostomy tube. In addition to a port which is used to attach the adaptor to a monitor, the adapter may further comprise at least one drainage port.

In another embodiment, the adapter is connected to a drainage port.

In another embodiment, at least one drainage port is in the tubing.

In yet another embodiment, at least one drainage port is in the port.

In an embodiment, at least one drainage port is adjacent to the tubing. In another embodiment, a plurality of drainage ports are adjacent to the tubing.

In a further embodiment, at least one drainage port is located before the tubing.

In another embodiment, at least one drainage port is located after the port.

In a further embodiment, at least one drainage port is located before the port. At least one drainage port helps relieve clogging of the tubing.

In a preferred embodiment, the patient is an infant or young child.

The present invention provides a medical device for monitoring breathing that comprises a) an adapter that slips onto the output of clinically used tracheostomy tubes and b) a monitor that houses an air flow sensor, control electronics and a battery.

### BRIEF DESCRIPTION OF THE DRAWINGS

So that those having ordinary skill in the art will have a better understanding of how to make and use the disclosed device, reference is made to the accompanying figures wherein:

FIG. 1 shows an embodiment of the tracheostomy tube adapter unit of the invention.

FIG. 2 shows an embodiment of the monitor unit connected to the adapter unit.

FIG. 3 shows a circuit diagram of the internal electronics.

### DETAILED DESCRIPTION OF INVENTION AND EMBODIMENTS

A tracheostomy is performed on a pediatric patient for different reasons; most are to resolve upper respiratory issues. The leading complications of a tracheostomy result in some obstruction to normal breathing such as decannulation or secretions clogging the tracheostomy tube (www.hopkinsmedicine.org/tracheostomy/about/complications.html). A recent article reports “The most common tracheostomy-related causes of death are accidental decannulation and blockage of the tracheostomy tube”, (Paediatr Respir Rev. 2006 September; 7(3):175-84).

Care of the patient after a tracheostomy is centered on ensuring the patient has an unobstructed airway. Dr. N. Mathur states: tubal obstruction “can be prevented with the appropriate preoperative tube care. Tube blockage is avoided with frequent suctioning and an awareness of the problem”, http://emedicine.medscape.com/article/873805-overview).

Accordingly, a device with the means to monitor breathing and alert caregivers to an emergency situation is critical to the well-being of tracheostomy patients. Currently, the only available airflow monitors are incorporated into heavy mechanical ventilators and are only useful when a patient is connected and restricted to the bed.

Accordingly, this invention relates to a device that monitors expiratory flow in patients with tracheostomies and will alarm when expiratory flow is low indicating a potential problem with either obstruction or dislodgement.

The breathing monitor is comprised of two parts:

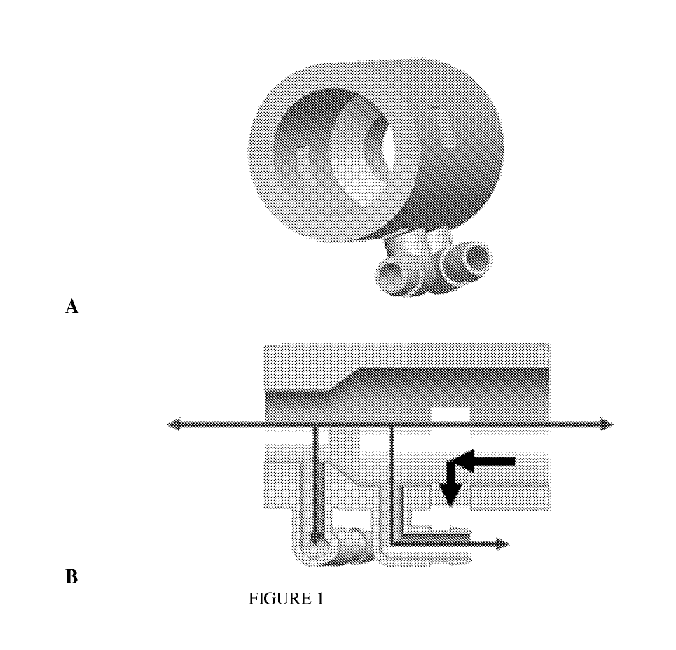

1) an adapter that slips onto the output of clinically used tracheostomy tubes, such as the exemplary adapter shown in FIG. 1; and

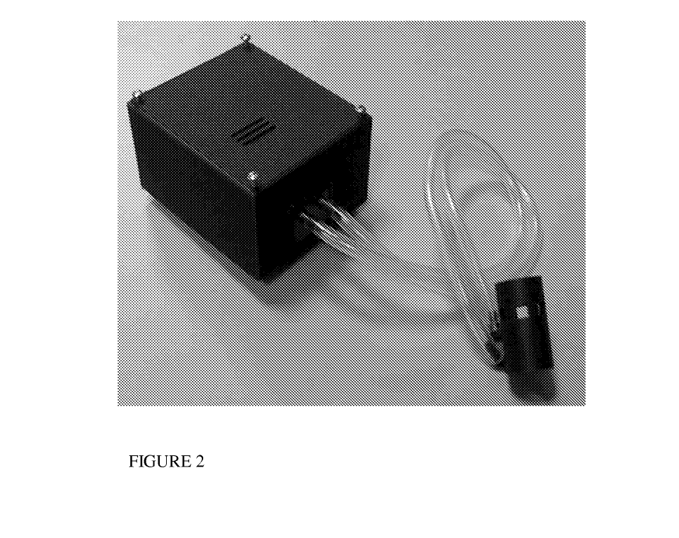

2) a monitor unit that houses the airflow sensor, control electronics, and battery, such as the exemplary monitor unit shown in FIG. 2.

The adapter piece extends from the outlet of the tracheostomy. It functions to direct flow over the two tubing input holes. One design consideration of the adapter is that tracheal secretions have to be diverted from entering the tubes. One solution is to provide drainage openings proximate to the tube inputs. This allows gravity to drain secretions before they can affect the sensor's readings. Tubing is used to transmit the signal to the inputs of the sensor within the electronics unit. Testing confirmed that this is a reliable and safe way to connect the sensor while minimizing extraneous material attached to the tracheostomy adapter.

The adapter can have one or more drainage openings, or alternatively, the drainage opening can be in the tubing or in the port. At least one drainage port may be adjacent to the tubing. Alternatively, a plurality of drainage ports are adjacent to the tubing. A drainage port may be located before the tubing, after a port or before a port. At least one drainage port helps relieve clogging of the tubing. It can be constructed of any appropriate material known in the art. Non-limiting examples include injection molded or extruded plastic.

The adapter unit is generally connected via two tubes to the monitor unit. The tubing connects to the two ports on the side of the adapter unit and directs pressure related to airflow to the monitor unit. The necking within the tube creates a pressure differential across the two ports that is sensed by a pressure differential airflow sensor in the monitor unit.

An example of an adapter of the invention is shown in FIG. 1, where A shows the full adapter and B shows a cross-section of the same adapter. The heavy black arrows indicate how mucosal secretions will flow through a drainage port in the tracheostomy adapter. The thinner arrows represent airflow from breathing.

The airflow sensor is chosen from those sensitive enough to detect the airflow rates associated with patient ventilation. It must also be constantly checking to determine the airflow associated with breathing and it must produce a signal sufficient to sound an alarm if sufficient airflow is not detected. In pediatric embodiments, the device must be sensitive enough to detect the low airflow rates that the infants produce.

The monitor unit also uses a compact and rechargeable microprocessor which can accept the signal from the airflow sensor that is in proportion to the pressure differential in the adapter unit (i.e. the faster the airflow, the larger the signal). The controller's output is connected to a alarm which can be alarmed to sound if there is no measurement from the sensor (no airflow) after a time delay. In addition a low-battery indicator can be included.

The device has been tested using a Bag-Valve-Mask; a manually controlled ventilation that mimics the natural respiratory volume and flow of an infant. The monitor unit housing and adapter unit were made using a 3-D rapid prototype printer out of ABS plastic. Alternative material for use in the adaptor can included, for example, hospital grade plastics such as polypropylene.

### Experimental

A prototype of the device of the invention was developed and tested as follows.

Adapter and Housing

An adapter as shown in FIG. 1 was constructed. For the prototyping process the materials used were chosen to show only the functionally of the device. The adapter piece and housing were created using a 3D printer and the tubing used not medical grade. For a future marketable product the manufacturing process would likely be an extruded or injection molded plastic housing and adapter piece. This streamline type of manufacturing would decrease the cost of the final product and therefore making the overall market price of the product lower.

The adapter was 9 mm in diameter at the narrow end and 15 mm in diameter at the wide end. The portal openings were 3 mm in diameter and the drainage opening was 5 mm by 5 mm The entire adapter was 36 mm long and the outside diameter at the wide end was 20.93 mm

The monitor housing was 77 mm by 90 mm and 47 mm tall.

Monitor Unit

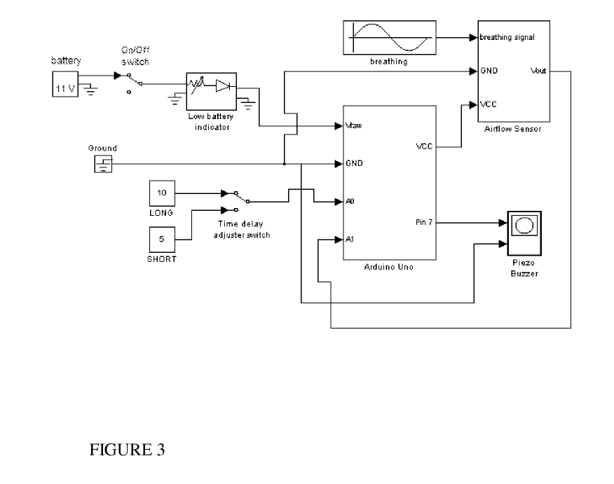

The components listed in Table 1 were purchased and assembled as described in FIG. 3.

TABLE 1

| Item | Description | Sku |
|---|---|---|
| Arduino Mini Pro 328 - | Microcontroller | DEV-09218 |
| 5 V/16 MHz | | |
| Arduino Uno | Microcontroller | DEV-09950 |
| JST RCY Connector - | Connecting ends of wires | PRT-10501 |
| Male/Female Set (2-pin) | | |
| 85 dB Piezo Buzzer | Audible Alarm component | CX 1606C |
| Mini Push Button Switch - SMD | Button to reset alarm | COM-08720 |
| | (tiny option) | |
| Momentary Push Button Switch | Button to reset alarm | COM-09190 |
| | (larger option) | |
| TENERGY 31003 LITHIUM LI- | Li-ion battery | TENERGY- |
| ION 18650 7.4 V 2200MAH | | 18650-2200-PK |
| BATTERY PACK | | |
| Breadboard Power Supply | Voltage regulator with barrel | PRT-00114 |
| 5 V/3.3 V | jack | |
| Wall Adapter Power Supply - | AC DC adapter (connects | TOL-00298 |
| 9 VDC 650 mA | from wall to barrel jack) | |
| CONN MOD JACK 6-4 RT/A | “Phone-jack” wire port | A31426-ND |
| PCB 50AU | (female) | |
| CONN JUNCT Box 4 POS 2D | Pitch Adapter | A29183-ND |
| FREE HANG | | |
| Sensor Airflow 200 SCCM Long | Zephyr Sensor | 480-3323-ND |
| 3.3 V | | |

### Testing

The prototype was tested for the efficiency of the electrical components meeting the operational voltage, interconnection, airflow rate detection range and physical design specifications. Additionally, the adapter did not need any additional modifications to the physical design since the unit fit perfectly into existing tracheostomy tube dimensions as well as, creating a tight seal and yet allowing for the required amount of air pressure to be detected via its pressure differential design. Due to the efficiency of the programmed microcontroller, the low battery alarm was verified. By using two different voltage supply ranges the functional order of the device was confirmed.

All variations and combinations of the features above are intended to be within the scope of the specification.

What is claimed is:

1. A medical device for monitoring breathing of a patient comprising an adapter with at least one port;
a monitor; and
at least one tubing connecting the at least one port and the monitor wherein the adapter attaches to a tracheostomy tube.

2. The medical device of claim 1, wherein the monitor comprises an air flow sensor, an electronic control and a battery.

3. The medical device of claim 1, wherein the monitor further comprises an alarm.

4. The medical device of claim 1, wherein the monitor is connected to an alarm.

5. The medical device of claim 1, wherein the adapter further comprises at least one drainage port.

6. The medical device of claim 1, wherein the adapter is connected to a drainage port.

7. An adaptor for connecting a tracheostomy tube to a medical device for monitoring breathing of a patient which comprises a housing that fits over the tracheostomy tube wherein the housing contains a port for connecting a tube attached to the medical device.

8. The adapter of claim 7 further comprising a drainage port.

9. The adapter of claim 7 which comprises 2 ports.

10. The adapter of claim 7 which comprises two ports and a drainage port.
